(19) United States
(12) Patent Application Publication
(10) Pub. No.: US 2025/0332387 A1
(43) Pub. Date: Oct. 30, 2025

(54) GUIDEWIRE TORQUE DEVICE AND METHOD OF USE

(71) Applicant: ABBOTT CARDIOVASCULAR SYSTEMS INC., Santa Clara, CA (US)

(72) Inventors: Matthew Gillick, Murrieta, CA (US); Joshua Jensen, Canyon Lake, CA (US); Stephanie Maldonado, Winchester, CA (US); Puneet Gill, Anahiem, CA (US)

(73) Assignee: ABBOTT CARDIOVASCULAR SYSTEMS INC., Santa Clara, CA (US)

(21) Appl. No.: 19/041,638

(22) Filed: Jan. 30, 2025

Related U.S. Application Data
(60) Provisional application No. 63/561,801, filed on Mar. 6, 2024.

(51) Int. Cl. A61M 25/09

(52) U.S. Cl. CPC A61M 25/09; A61M 2025/09116; A61M 2205/586

(57) ABSTRACT

A torque device for torquing and advancing a guidewire into a patient's vascular system. The torque device is configured for single-handed use and can be used for gripping the guidewire to provide axial and rotational movement to the guidewire.

### CROSS-REFERENCES TO RELATED APPLICATIONS

This application claims priority from U.S. Application No. 63/561,801, filed Mar. 6, 2024 incorporated by reference in its entirety.

### BACKGROUND

Medical guidewires are commonly used for a variety of medical procedures. Such procedures include angioplasty, stenting, pacemaker insertion, electrophysiology studies, atherectomy, and thrombolysis and other coronary and peripheral endovascular procedures, and in endourology and therapeutic endoscopy of the gastrointestinal system. To position a guidewire at a desired location within a patient a medical professional navigates the guidewire through the patient's anatomy by manipulating the guidewire. Such manipulation includes advancing of the guidewire into a patient's vasculature or other portion of the patient's body while torqueing the guidewire. Torqueing the guidewire allows the medical professional to change the spatial orientation of the tip of the guidewire when negotiating tortuous turns and branches in the patient's vasculature such as the coronary arteries, or other relevant portion of the patient's anatomy.

To manipulate the guidewire, medical professionals have traditionally used devices which require two-handed operability. As the guidewire is advanced into the patient's artery, the distance between the patient's body and the torque device decreases. When the proximity between the patient's body and the torque device decreases, the medical professional will loosen the torque device, reposition the torque device proximally along the guidewire to provide an additional length of guidewire between the patient's body and the torque device, and then tighten the torque device to secure its position along the length of the guidewire. The process of loosening and repositioning the torque device may be repeated several times during the placement of the guidewire.

Many of the commercially-available torque devices require two-handed operability to loosen and tighten the device. Due to the complexities of some guidewire placement procedures, it can be inconvenient or impractical for a practitioner to utilize both hands to thread the guidewire through the catheter or reposition the torque device along the length of the guidewire. As a result, additional care and attention are required when manipulating the torque device relative to the guidewire during the procedure. This can lengthen the amount of time and the degree of difficulty necessary to complete the guidewire placement procedure. Additionally, traditional devices are often not adequately intuitive leading to misuse of the device and inadvertent damage to the guidewire. These devices can require specialized training to facilitate proper usage of the device and can still result in inadvertent misuse of the device during the course of the procedure. Additionally, some devices do not provide adequate gripping of the guidewire as may be required to push the guidewire through a calcified vascular lesion or other guidewire path occlusion. Where an occlusion is encountered, the practitioner may over tighten the device in a manner that causes damage to the guidewire such as kinking the wire or damaging a coating on the wire.

Generally, guidewires have a lubriquous or hydrophilic coating on the distal portion of the wire and a hydrophobic coating (PTFE) on the proximal portion of the wire to provide lubricity to permit the guidewire to pass more easily through a blood vessel. However, due to the lubricity, sufficient torque cannot be applied by simply rolling or twisting the proximal end of the guidewire by the clinician. Consequently, a torque apparatus is needed to grip the guidewire having a hydrophilic coating for adequate torque application without damaging the coating. When the clinician needs to reposition the torque apparatus along the guidewire, the user grasps one end of the torque apparatus while actuating a mechanism to release the guidewire with the other hand. The torque apparatus is then moved along the guidewire to reposition the torque device along the guidewire. As a result of the two-handed operation required to release the guidewire and reposition the torque apparatus, another clinician is needed to hold the guidewire steady while the torque device is repositioned, all the while being careful to not damage the coatings on the guidewire. Additionally, when repositioning the torque device the physician releases the guidewire while trying to position the torque device, which can easily result in losing the wire position in the patient or even slide out and get contaminated.

Physicians and patients would benefit from a single-handed torque device that would allow the physician to quickly position the torque device with one hand and rotate (torque) and advance the guidewire to facilitate penetrating a calcified lesion.

### SUMMARY OF THE INVENTION

Multiple embodiments are disclosed herein relating to a guidewire torque device which allows the physician to use the torque device with one hand to simultaneously torque and advance the guidewire through the patient's vasculature. The torque devices disclosed herein are used for gripping or securing and releasing a guidewire to permit rotational manipulation and longitudinal advancement of the guidewire to more easily penetrate a calcified lesion in a vessel or other tortuous anatomy. In some embodiments disclosed herein, the torque device is configured for single handed operation by the physician. In other embodiments, the physician can use the torque device to rotate (torque) the guidewire clockwise or counterclockwise, while advancing the guidewire distally in small increments without the need to manually manipulate the guidewire.

In one embodiment, a guidewire torque device provides for both rotation in either direction and translation (tapping effect) of the guidewire at the same time. The feature of translation (distal movement) of the guidewire allows an easier and faster (more iterations) penetration of a calcified lesion. The torque device works by using a ratcheting mechanism that simultaneously pushes the guidewire distally and rotates the guidewire (in either direction).

### BRIEF DESCRIPTION OF THE DRAWINGS

FIG. 1 is an elevational view of a torque device for use in gripping and manipulating a guidewire.

FIG. 2A is an exploded perspective view of the torque device of FIG. 1.

FIG. 2B is a front view of a collet shown in FIG. 2A.

FIG. 2C is a side view of the collet in FIG. 2B.

FIG. 2D is an elevation view of a cap.

FIG. 2E is a cross-sectional view of the cap of FIG. 2D.

FIG. 3A is a front elevational view depicting an actuator and first gear housing.

FIG. 3B is a top view depicting the actuator and first gear housing of FIG. 3A.

FIG. 3C is a perspective view depicting the actuator and first gear housing of FIG. 3A.

FIG. 4A is a front elevational view depicting actuator grip bars.

FIG. 4B is a top view depicting the actuator grip bars of FIG. 4A.

FIG. 4C is a perspective view depicting the actuator grip bars of FIG. 4A.

FIG. 5A is a front elevational view depicting a main housing.

FIG. 5B is a longitudinal cross-sectional view taken along lines 5B-5B depicting the main housing of FIG. 5A.

FIG. 5C is a top view depicting the main housing of FIG. 5A.

FIG. 6A is a front elevational view depicting a second gear housing.

FIG. 6B is a longitudinal cross-sectional view taken along lines 6B-6B depicting the second gear housing of FIG. 6A.

FIG. 6C is a bottom view depicting the second gear housing of FIG. 6A.

FIG. 6D is a top view depicting the second gear housing of FIG. 6A.

FIG. 7A is a front elevational view depicting a spring housing.

FIG. 7B is a longitudinal cross-sectional view taken along lines 7B-7B depicting the spring housing of FIG. 7A.

FIG. 7C is a top view depicting the spring housing of FIG. 7A.

FIG. 7D is a bottom view depicting the spring housing of FIG. 7A.

FIG. 7E is a perspective view depicting the spring housing of FIG. 7A.

FIG. 7F is a front elevational view depicting a spring for insertion into a cavity in the spring housing of FIG. 7A.

FIG. 8A is a perspective view, partially in section, depicting a guidewire torque device having a squeeze lever to activate rotational and axial movement to the guidewire.

FIG. 8B is a cross-sectional view taken along lines 8B-8B depicting the handle and squeeze lever of the guidewire torque device of FIG. 8A.

FIG. 9 is a top view depicting the guidewire torque device of FIG. 8A.

FIG. 10A is a partial cross-sectional view taken along lines 10A-10A depicting the guidewire torque device of FIG. 8A.

FIG. 10B is a cross-sectional view taken along lines 10B-10B depicting the handle and springs of the guidewire torque device.

FIG. 10C is a cross-sectional view taken along lines 10C-10C depicting the squeeze lever of the guidewire torque device.

FIG. 11 is a partial cross-sectional view taken along lines 11-11 depicting the squeeze lever moved into the handle and causing the collet and guidewire to rotate and move distally.

FIG. 12A is a partial elevational view depicting uniform spacing of helical grooves in the drive screw.

FIG. 12B is a partial elevational view depicting non-uniform spacing of helical grooves in the drive screw.

FIG. 12C is a partial elevational view depicting helical grooves in a non-round drive screw.

FIG. 12D is a partial elevational view depicting irregular-spaced helical grooves in the drive screw.

FIG. 13A is a partial elevational view depicting spur gears for use in moving the guidewire axially.

FIG. 13B is a partial elevational view depicting spur gears for use in moving the guidewire axially.

### DETAILED DESCRIPTION OF THE PREFERRED EMBODIMENTS

Embodiments disclosed herein relate to a medical guidewire torque device which offers advantages which are not currently available in prior art devices. The torque devices disclosed herein are used for attaching to and selectively gripping or securing and releasing a guidewire to simultaneously provide rotational and longitudinal advancement of the guidewire to steer the guidewire through a vessel or series of vessels or other tortuous anatomy. In some embodiments disclosed herein, the torque device can be used by the physician using only one-handed operation. The torque device provides an advantage to the physician in manipulating the guidewire in tortuous anatomy.

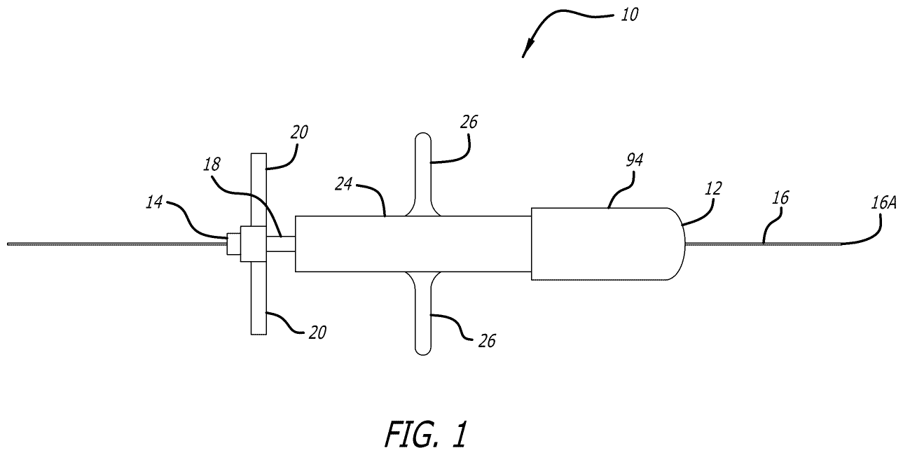

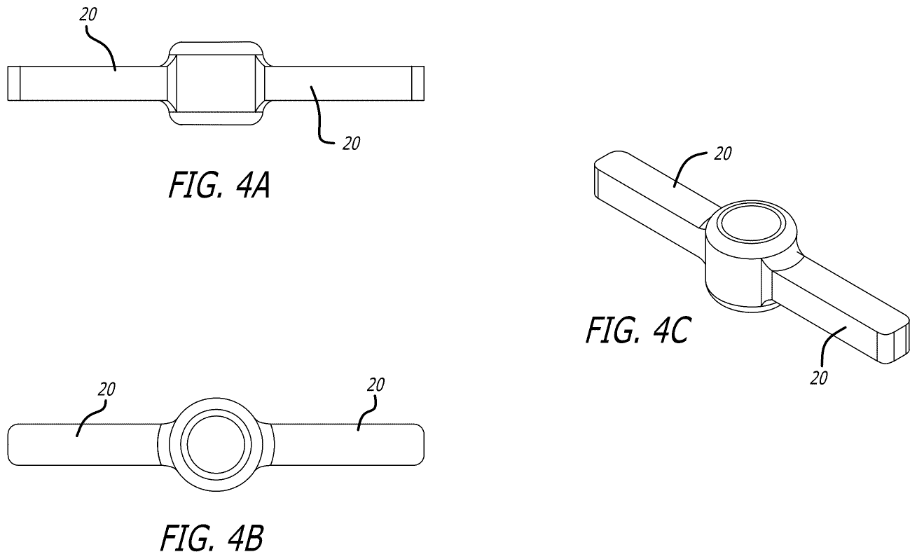

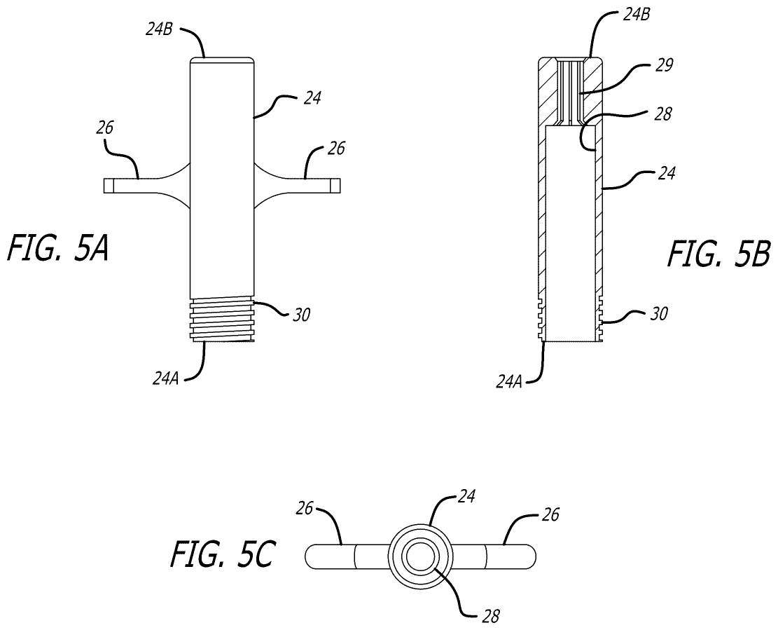

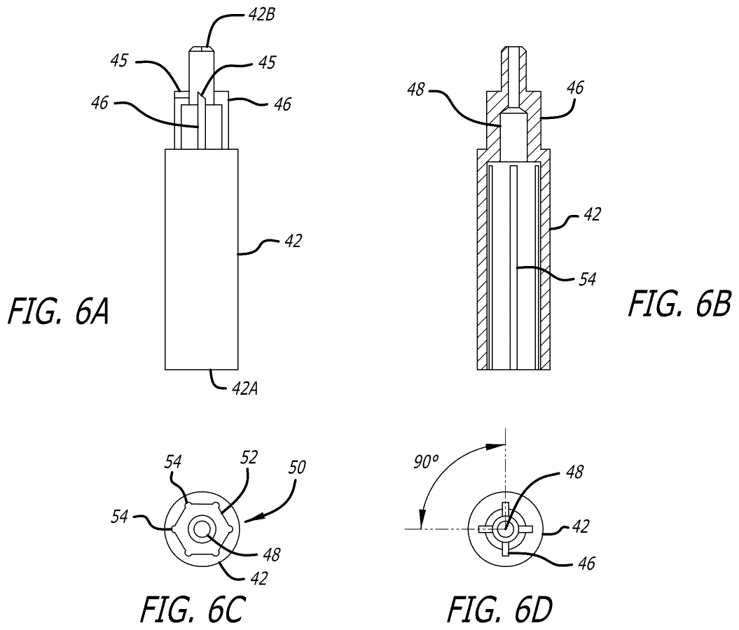

In one embodiment, as shown in FIGS. 1-7E, a torque device 10 has a distal end 12 and a proximal end 14. The torque device 10 is comprised of an assembly of parts including an actuator 18, a main housing 24, a first gear housing 32, a second gear housing 42, a collet housing 60, a collet 70, a cap 84, and a spring housing 94. Each of the parts has a lumen through which a guidewire 16 extends and can be torqued and advanced distally. The actuator 18 has a distal end 18A and a proximal end 18B and is positioned for slidable movement in main housing 24, but not for rotational movement. The actuator 18 has grip bars 20 extending transverse to a longitudinal axis of the actuator 18. The actuator 18 has a lumen 22 extending therethrough. The main housing 24 has a distal end 24A and a proximal end 24B and a lumen 28 extending therethrough. The main housing 24 has a pair of grip bars 26 extending transverse to a longitudinal axis of the main housing. The main housing 24 has external threads 30 adjacent the distal end 24A. As shown in FIG. 5B, at the proximal end 24B of the main housing 24, a plurality of grooves 29 are formed in the main housing lumen 28. The plurality of grooves are spaced apart 45° from each other. While the 45° spacing is preferred, the plurality of grooves 29 can be spaced apart at different angles including 20°, 30°, 60° or 75°.

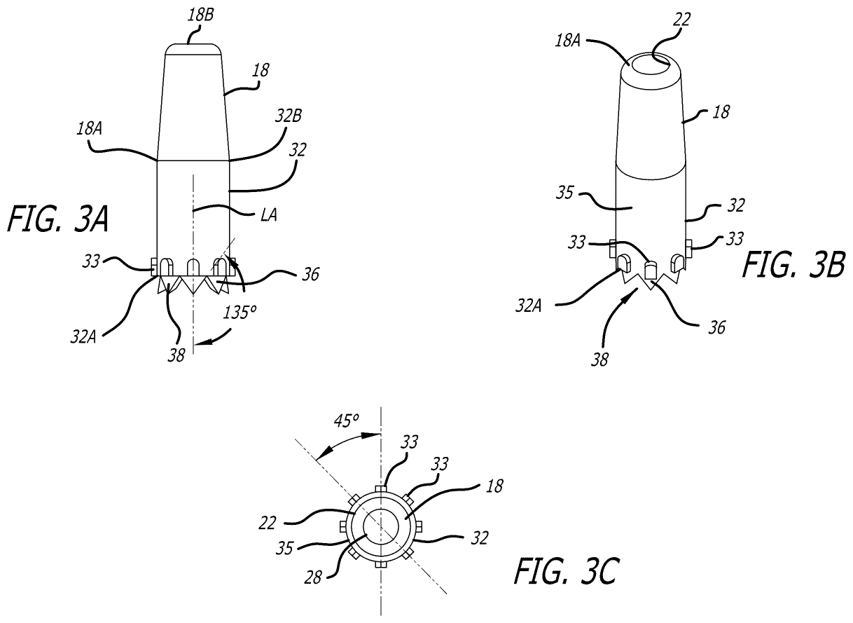

As further shown in FIGS. 3A-3C, a first gear housing 32 has a distal end 32A and a proximal end 32B and a lumen 40 extending therethrough. The first gear housing 32 has a plurality of raised tabs 33 that project radially outward from the outer surface 35 of the first gear housing 32. In one embodiment, the tabs 33 are spaced apart 45°, however, in other embodiments the spacing between the tabs 33 can be 20°, 30°, 60° or 75°. A first gear 34 is attached to the distal end 32A of the first gear housing 32 and can be a separate part or preferably formed as a unitary structure with the first gear housing 32. The first gear 34 has first teeth 36 that have first gear teeth ends 38 that are angled 135° relative to a longitudinal axis (LA) of the teeth 36. In other embodiments, the first gear teeth ends are angled in a range from 75° to 160°. In one embodiment the first gear teeth 36 are spaced apart 45°, however, in other embodiments the spacing between the teeth 36 can be 20°, 30°, 60° or 75°. A second gear housing 42 has a distal end 42A and a proximal end 42B and a lumen 48 extending therethrough. The second gear housing 42 has second gear teeth in the form of elongated splines 46, wherein each spline has teeth 44 that have second gear teeth ends 45 that are angled 135° relative to a longitudinal axis (LA) of the splines 46. In other embodiments, the second gear teeth ends are angled in a range from 75° to 160°. In one embodiment, the elongated splines 46 (and hence the second gear teeth 44) are spaced apart 90°, however, in other embodiments the spacing between the elongated splines 46 can be 30°, 45°, 60° or 120°. The second gear housing lumen 48 has a non-round transverse cross-section 50 extending from the distal end 42 toward the proximal end 42B for a substantial portion of the lumen 48. The non-round transverse cross-section 50 can be any polygonal shape such as a pentagon, octagon, or more preferably a hexagonal-shaped lumen 52. In one preferred embodiment, six grooves 54 are formed in each of the corners of the hexagonal-shaped lumen 52

In one embodiment, shown in FIGS. 3A-5C, the actuator 20 and the first gear housing 32 are slidably positioned into the main housing lumen 28 so that the plurality of raised tabs 33 align with and slide in the plurality of grooves 29 at the proximal end 24B of the main housing 24. Thus, the actuator 20 and the first gear housing 32 can slide distally and proximally relative to the main housing 24, but neither can rotate relative to the main housing because the plurality of tabs 33 slide in the plurality of grooves 29 and prevent rotational movement.

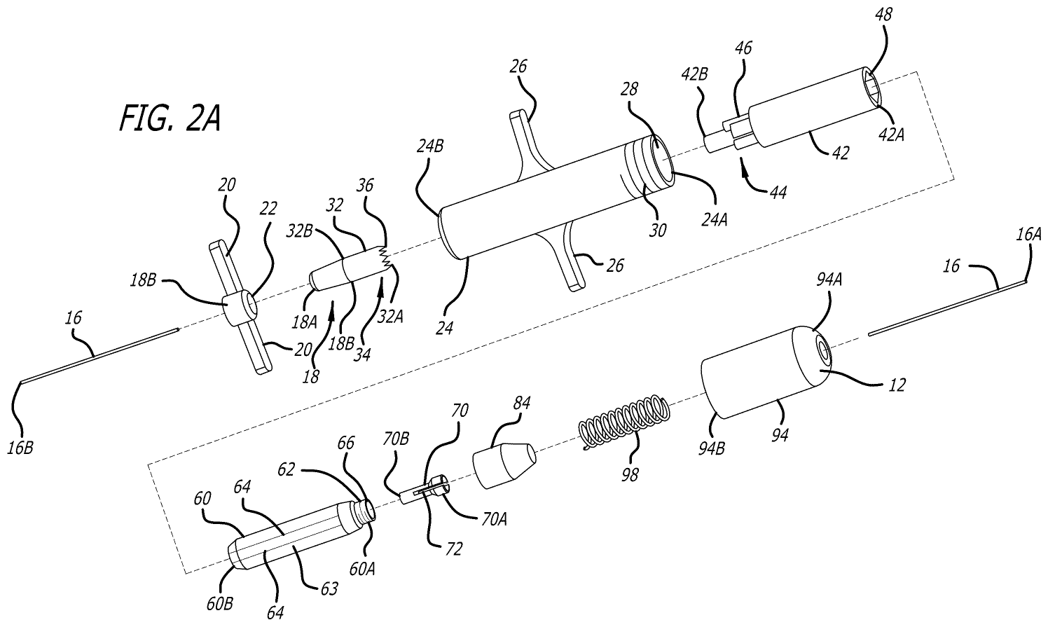

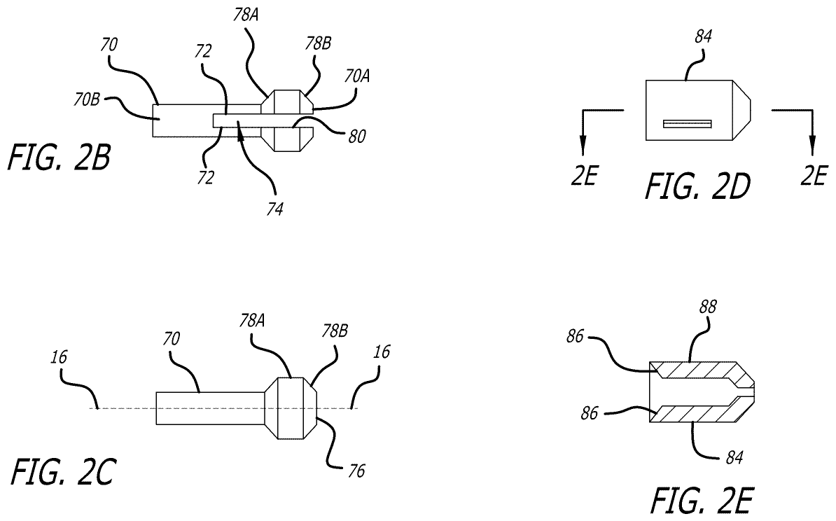

With further reference to FIGS. 2A-2E, a collet housing 60 has a distal end 60A, a proximal end 60B, and external threads 62 on the distal end 60A. A plurality of outer ridges 64 extend longitudinally along an outer surface 63 of the collet housing 60. The outer ridges 64 are preferably spaced apart 60° from each other, however, the outer ridges 64 can be spaced apart at different angulations including 30°, 45°, 75° or 90°. When assembled, the proximal end 60B is inserted into the distal end 42A of the second gear housing 42. The plurality of outer ridges 64 mesh with and slide into the grooves 54 in the second gear housing 42. Thus, the collet housing 60 can slide within the second gear housing 42, but cannot rotate relative thereto. The collet housing 60 has a lumen 66 extending therethrough and there is a tapered portion 68 of the lumen 66 at the distal end 60A of the collet housing 60.

In one embodiment, as shown in FIGS. 2A-2E, a torque device 10 is used to grip and manipulate a guidewire 16 using single handed operation. The torque device 10 includes a collet housing 60 and a cap 84. The collet housing 60 has external threads 62 and cap 84 has internal threads 88 so that the cap can be screwed onto the collet housing. A collet 70 is enclosed in the collet housing 60 and is retained inside the collet housing after the cap 84 is screwed onto the external threads 62. The collet 70 has a plurality of longitudinally extending fingers 72 that are spring biased towards an open position 74. When the fingers 72 are in a compressed and in a closed position 76, the fingers 72 will grip the guidewire 16 and securely retain it for manipulation and advancing into the vascular system. The collet 70 has a first tapered face 78A and a second tapered face 78B at the collet distal end 70A. The collet housing 60 has a tapered portion 68 that slides on the first tapered face 78A of the collet 70. As the cap 84 is screwed onto the collet housing 60, the second tapered face 78B of the collet 70 engages a tapered surface 86 on the cap 84 and slides along the tapered surface 86 of the cap 84, which in turn compresses the plurality of fingers 72 onto the guidewire, thereby gripping the guidewire 16 so that the torque device can move the guidewire without sliding along the longitudinal surface of the guidewire as the cap 84 is screwed onto the collet housing 60, a force vector is generated by the second tapered face 78B of the collet 70 sliding along the tapered surface 86 of the cap 84. The force vector overcomes the spring bias of the plurality of fingers, which are spring biased toward the open position 74. A gripping surface 80 on the collet 70 is forced onto the guidewire 16 to securely grip the guidewire so that the torque device 10 can then be used by the physician to manipulate the guidewire. The plurality of fingers 72 on the collet 70 spring apart to the open position 74 when the cap 84 is unscrewed from the collet housing 60, to cause the gripping surface 80 on the collet 70 to release the guidewire 16.

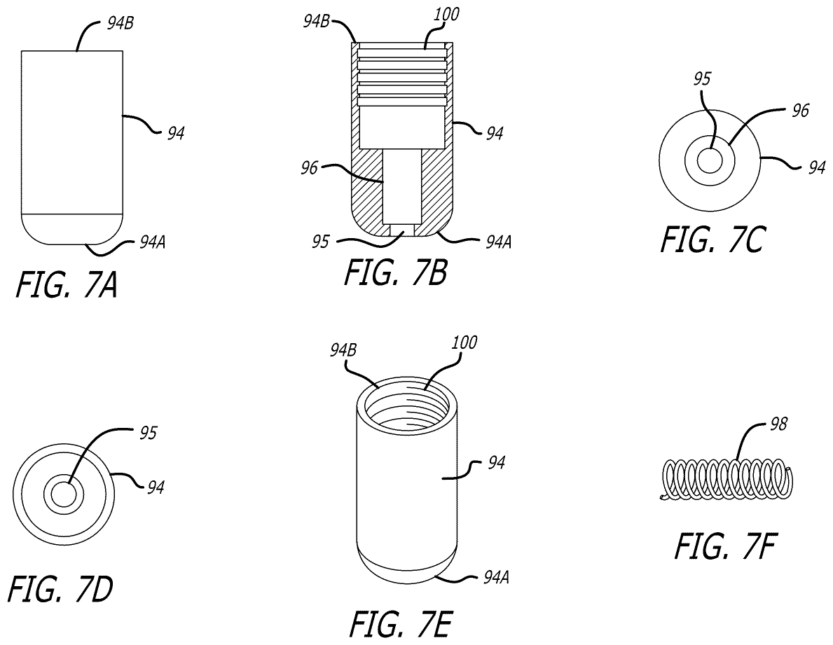

With reference to FIGS. 7A-7E, a spring housing 94 is located at the distal end 12 of the torque device 10 and has a cavity 96 sized for receiving a spring 98. The spring housing 94 has a distal end 94A, a proximal end 94B, a lumen 95 extending therethrough, and internal threads 100 on the proximal end 94B. When assembled, the spring housing 94 is attached to the second gear housing 42 by screwing the external threads 43 on the second gear housing 42 onto the internal threads 100 on the spring housing 94.

The method of using the torque device 10 requires some assembly by the physician when treating the patient. Once the physician has advanced the guidewire 16 into the patient's vascular system, typically a coronary artery, the distal end of the guidewire may encounter difficulty in crossing or penetrating a tight lesion, calcified plaque, or a chronic total occlusion (CTO). The torque device 10 can be used to not only torque the guidewire, but to provide a tapping effect to move the guidewire 16 distally. The physician can click the torque device 10 to provide multiple, quick iterations of advancing and withdrawing the guidewire 16 relative to the torque device, while simultaneously torquing the guidewire 16.

More specifically, the proximal end 16B of the guidewire 16 is inserted through the distal end 70A of the collet 70 and the torque device 10 is advanced over the guidewire 16 until the torque device is positioned close to the patient where the guidewire has entered the patient's body. The cap 84 is screwed onto the main housing and when the cap 54 is tightened, it closes the fingers 72 on the collet 70 so that the fingers 72 firmly grip the guidewire 16. The spring housing 94 is next screwed onto the second gear housing 42 so that the guidewire 16 now extends all the way through the torque device 10.

To torque and advance the guidewire 16, the physician can use one or both hands to repeatedly push the actuator 18 distally which causes the first gear teeth 36 to engage the second gear teeth 44. The first gear teeth ends 38 are angled at 135° and the second gear teeth are angled at 135° which results in the second gear housing 42 to rotate 45° relative to the actuator 18 and the first gear housing 32. Rotation of the second gear housing 42 also causes the collet 70 to rotate and since the collet 70 is gripping the guidewire 16, the guidewire 16 also rotates 45°. Simultaneous with the rotation, the actuator 18 is moving distally and it pushes the collet housing 60 distally, which in turn moves the collet 70 distally. As the collet 70 moves distally so does the guidewire 16. Further, since the collet 70 is fixed inside of the collet housing 60, which is slidably mounted in the second gear housing 42, the collet 70 advances distally along with the collet housing to compress the spring 98 inside the spring housing 94. The spring 98 is based toward the open position so as the actuator 18 is pushed distally by the physician, the spring force is overcome and the spring 98 is compressed. The distal end 16A of the guidewire moves distally into the lesion or CTO and simultaneously rotates upon activation of the actuator 18. In one embodiment, a single activation of actuator 18 results in the guidewire 16 moving distally in a range from 0.0787 inch (2 mm) to 0.3937 inch (10 mm). In another embodiment, the guidewire 16 moves distally in a range from 0.0394 inch (1 mm) to 0.5906 inch (15 mm). The torque device 10 can be modified to provide more or less torque (45°) and distal movement to the guidewire 16.

When the physician releases the actuator 18, the spring 98 expands to its open position and pushes the collet 70, collet housing 60, second gear housing 42, and the actuator 20 proximally. A rapid pushing and releasing the actuator 18 provide a tapping effect on the guidewire distal end 16B and thereby providing a quicker and more efficient penetration of the lesion or CTO.

As shown in FIGS. 1, 2, 4A-4C, and 5A-5C, the actuator 18 has actuator grip bars 20 and the main housing 24 has main housing grip bars 26. The physician can squeeze and release the actuator grip bars 20 and the main housing grip bars 26 toward and away from each other to depress and release the actuator 18 rapidly as discussed herein.

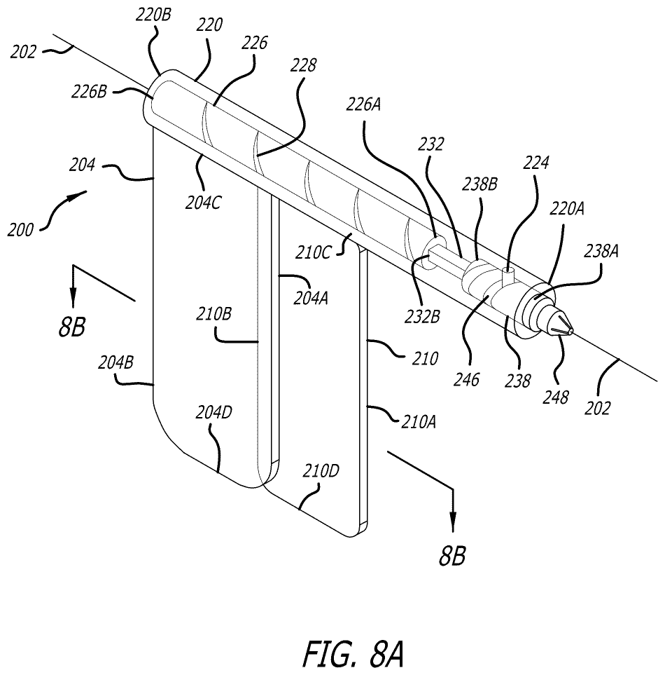

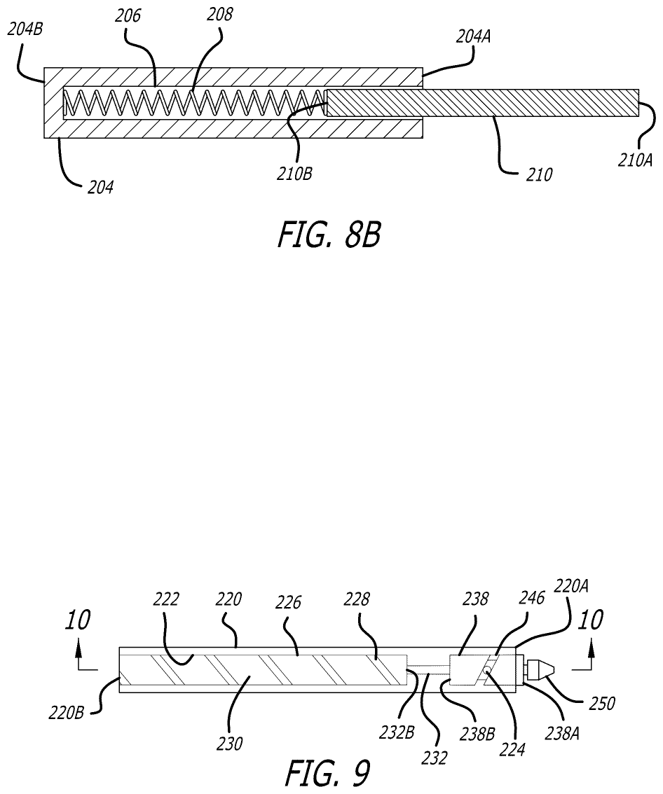

In another embodiment, shown in FIGS. 8A-11, a guidewire torque device 200 has a distal end 200A and a proximal end 200B, and a guidewire 202 for insertion therein. A handle 204 has a distal end 204A, a proximal end 204B, a top 204C and a bottom 204D. The handle 204 has a cavity 206 that is configured to retain one or more springs 208. Multiple spring retainers 207 can be mounted in the cavity 206. The springs 208 can be in the form of a coil spring, a leaf shaped spring, or any other shaped spring known in the art. In one embodiment, the handle 204 is rectangular-shaped and is hollow so that the cavity 206 also is rectangular-shaped. A squeeze lever 210 has a distal end 210A, a proximal end 210B, a top 210C, and a bottom 210D. In one embodiment, the squeeze lever 210 is rectangular-shaped and is sized for slidable insertion into the rectangular-shaped cavity 206 of the handle 204. The squeeze lever 210 has a top rail 212A and a bottom rail 212B that are configured to slide in a top channel 214A and a bottom channel 214B in the cavity 206 of the handle 204. The squeeze lever 210 has an open position 215A and a closed position 215B. A screw drive pin 218 extends from the top 210C of the squeeze lever 210.

The top 204C of the handle 204 is attached to a housing 220 having a distal end 220A and a proximal end 220B. Preferably, the housing 220 is cylindrical in shape and has a lumen 222 extending therethrough. A cam pin 224 is attached to the housing 220 adjacent the distal end 220A and extends into the housing lumen 222. The housing lumen 222 is sized to receive a cylindrically-shaped drive screw 226 having a distal end 226A and a proximal end 226B. A helical-shaped groove 228 is formed in an outer surface 230 of the drive screw 226 and it extends for a portion along the outer surface 230. The screw drive pin 218 on the top 210C of the squeeze lever 210 is configured for slidable movement in the helical groove 228 of the drive screw 226. A guidewire lumen 231 extends through the drive screw 226.

A spline shaft 232, preferably a square-shaped solid shaft, has a distal end 232A and a proximal end 232B. The proximal end 232B of the spline shaft 232 is attached to the distal end 226A of the drive screw 226. A guidewire lumen 234 extends through the spine shaft 232. In one embodiment, the drive screw 226C and the spline shaft 232 are formed as a unitary structure by molding a high strength polymer. The distal end 232A of the spline shaft 232 is sized for slidable insertion into a square-shaped lumen 240 extending through a barrel cam 239. The barrel cam 238 is preferably cylindrical and has a distal end 238A and a proximal end 238B and is positioned in housing lumen 222 adjacent the distal end 220A of the housing 220. The barrel cam 238 is sized for rotational and slidable movement relative to the housing 220. A guidewire lumen 242 extends through the barrel cam 238. The barrel cam 238 has an outer surface 244 having a helical groove 246. The helical groove 246 is sized to receive the cam pin 224 attached to the housing 220 near the distal end 220 of the housing.

A guidewire collet 248 is inserted into the distal end 238A of the barrel cam 238. The collet has a plurality of fingers for gripping the guidewire 202 so it cannot move relative to the torque device 200. The collet 248 in this embodiment (FIGS. 8A-10A) is identical in structure and operation to the collet 70 and cap 84 shown in FIGS. 2A-2C and described in the disclosure relating thereto. The collet 248 has a guidewire lumen extending therethrough. The plurality of fingers are biased toward an open position and when gripping the guidewire 202, the plurality of fingers are in a closed position. A cap 250 has a distal end, a proximal end, and internal threads adjacent the proximal end.

The embodiment of the guidewire torque device 200 shown in FIGS. 8A-12D can be operated by the physician using one or both hands to simultaneously rotate (torque) and axially move the guidewire 202 relative to the torque device 200. The torque device 200 is mounted onto the proximal end of the guidewire 202 by first inserting the guidewire proximal end into the cap 250 and then through the collet guidewire lumen 254, the barrel cam guidewire lumen 242, the square spline guidewire lumen 234, and the drive screw guidewire lumen 231. In one embodiment, the torque device 200 is advanced distally over the guidewire 202 until it is in a range from 0.39 inch (1.0 cm) to 7.87 inch (20.0 cm), from the patient. This distance can vary depending on physician preferences. In order to grip the guidewire 202, the cap 250 is screwed onto the barrel cam 238 causing the fingers (biased open) to tighten to a closed position onto the guidewire so that the torque device 200 firmly grips the guidewire 202.

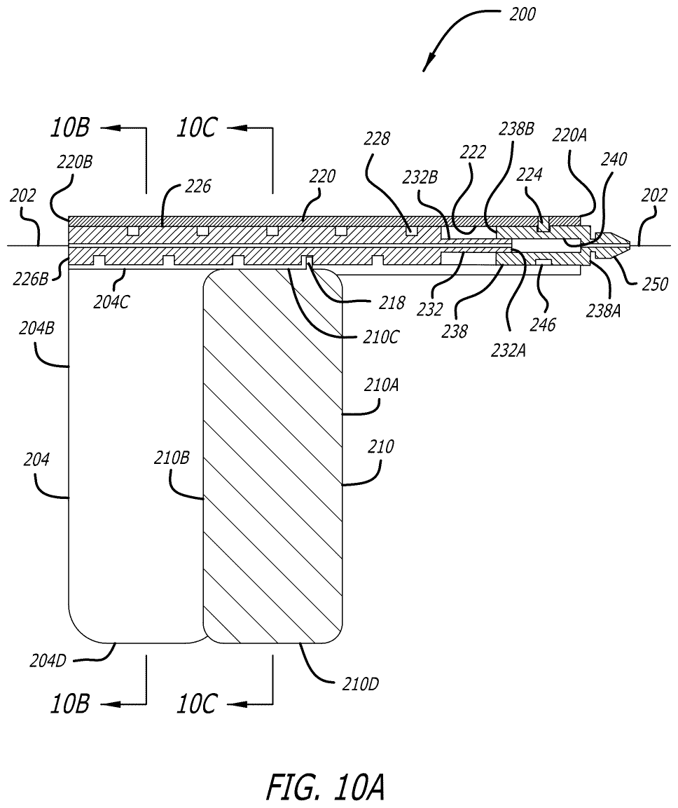

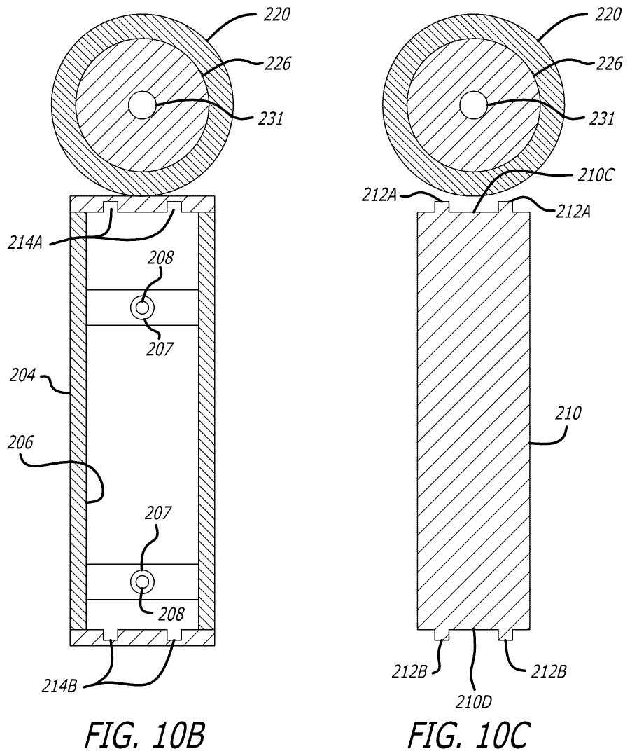

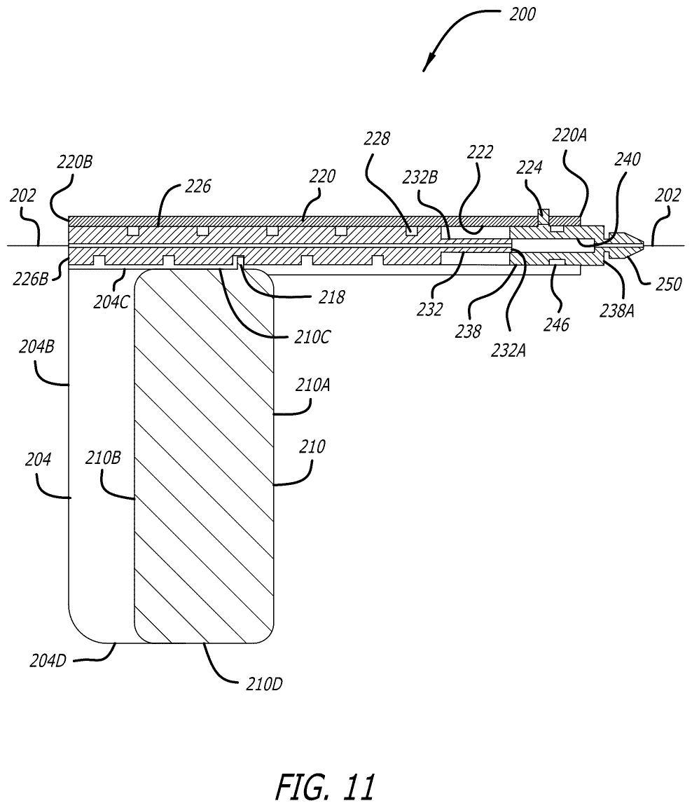

As shown in FIGS. 10A and 11, as the physician squeezes the squeeze lever 210 to move it proximally and slide into the cavity 206 of the handle 204, the one or more springs 208 are compressed. Further, the screw drive pin 218 moves along in the helical groove 228 of the drive screw 226 causing the drive screw 226 to rotate. The drive screw 226 can rotate clockwise or counterclockwise depending upon the shape of the helical groove 228, but importantly, the drive screw 226 does not move axially. Since the square spline shaft 232 is attached to the drive screw 226, it rotates the same number of degrees as the drive screw 226. Importantly, the square spline shaft 232 does not move axially. As the square spline shaft 232 rotates, it causes the barrel cam 238 to rotate the same number of degrees. As the barrel cam 238 rotates, the cam pin 224 slides along the helical groove 246 in the barrel cam 238. Since the cam pin 224 is fixedly attached to the housing 220, the barrel cam 238 moves distally relative to the square spline shaft 232. In other words, the square-shaped lumen 240 of the barrel cam 238 slides axially relative to the axially stationary square spline shaft 232. Thus, the barrel cam 238 rotates and simultaneously moves axially in a distal direction. FIG. 10A shows the barrel cam 238 inside the housing 220, and FIG. 11 shows the barrel cam 238 after it has moved distally a short distance from the distal end 220A of the housing 220. Since the collet 248 is firmly gripping the guidewire 202 and is inserted into the barrel cam 238, the collet 248 and guidewire 202 rotates the same number of degrees and also move the same distance distally as the barrel cam 238. In one embodiment, the torque device 206 can rotate the guidewire 202 from 5° to 360° and move the guidewire 202 distally from 0.0393 inch (1.0 mm) to 0.7874 inch (20.0 mm). The physician can squeeze the squeeze lever 210 its full range of motion or make repeated tapping or short squeezing motions to rotate and distally advance the guidewire 202 into contact with the calcified lesion in the patient's vessel (e.g., coronary artery).

When the physician releases the squeeze lever 210, the one or more springs 208 elongate thereby pushing the squeeze lever 210 distally in the cavity 206 of the handle 204. The top rail 212A and the bottom rail 212B slide in the respective top channel 214A and the bottom channel 214B to keep the squeeze lever 210 on track and provide a good tactile feel for the physician. As the squeeze lever 210 moves distally, the rotation and axial movement of the various parts reverses and returns to the starting position for further iterations. Repeated, short squeezing and releasing of the squeeze lever 210 provides a tapping effect on the distal end of the guidewire 202 to more easily penetrate a calcified lesion or CTO. The torque device 200 can be repositioned on the guidewire or removed from the guidewire by unscrewing the cap 250 to release the grip on the guidewire.

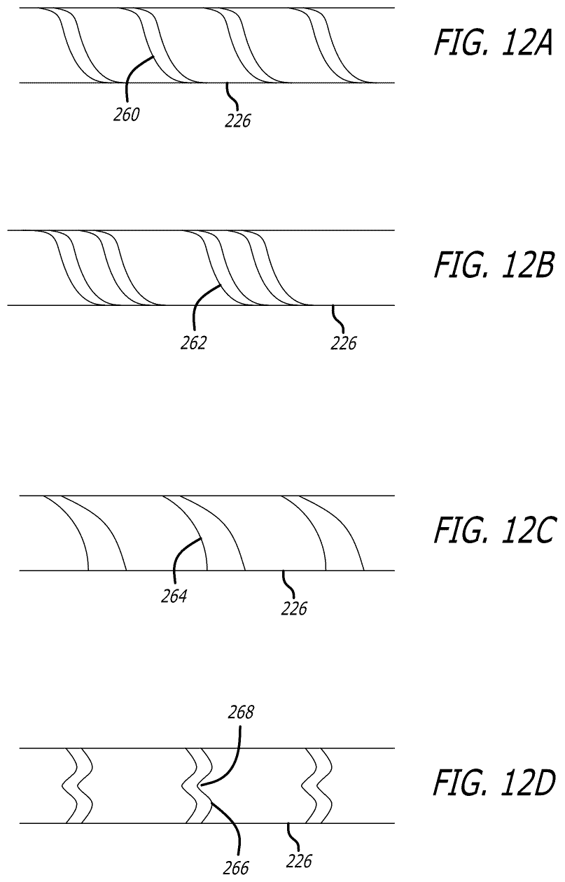

The pitch of the helical groove 228 in the drive screw 226 can vary to provide more or less rotation or either clockwise or counterclockwise rotation. Different helical groove patterns are shown in FIGS. 12A-12D. The helical groove 260 in FIG. 12A has uniform spacing between the grooves. The helical groove 262 in FIG. 12B has variable spacing between the grooves resulting in the aforementioned tapping effect. In FIG. 12C, the shape of the drive screw 226 changes the shape and pitch of the helical groove 262. For example, the drive screw 226 in FIG. 12C could be slightly elliptically-shaped, thereby forming a more elliptically-shaped helical groove 264. In FIG. 12D, a helical groove 266 has notches 268 that create both rotational and some axial movement.

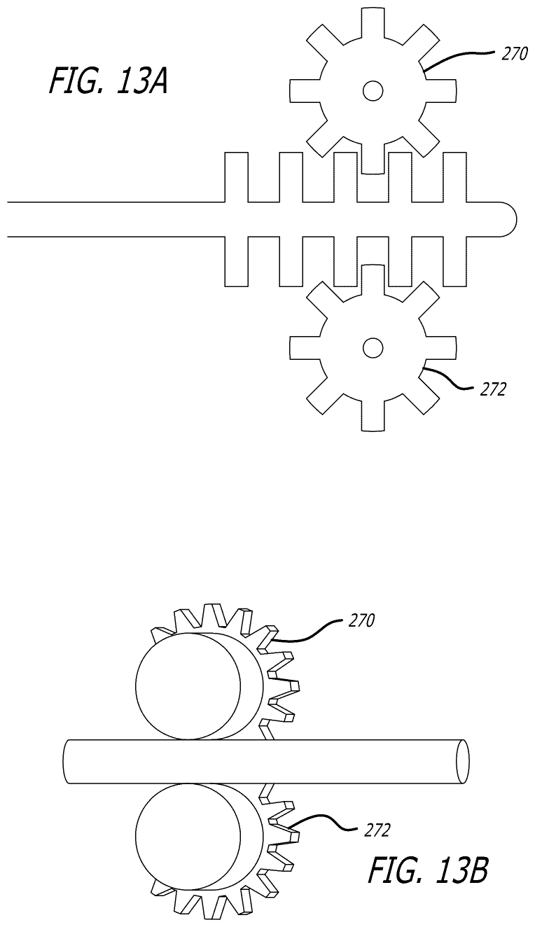

In one alternative embodiment, shown partially in FIGS. 13A and 13B, a pair of gears are associated with the handle and squeeze lever to impart axial movement to the guidewire. A first gear 270 can be mounted on the stationary handle and a second gear 272 can be mounted on a squeeze lever. As the squeeze lever is squeezed to move proximally, the second gear 272 rotates and moves axially while the first gear 270 rotates, but remains stationary. The guidewire is positioned in between the gears and moves axially with the axial movement of the second gear 272. Each gear can be a spur gear that is known in the art.

Most of the structure for the torque device 10 and 200 can be formed from plastic or polymer materials that are well known in the art. The collet 70 and 248 typically are made from brass and the springs 208 are metallic as known in the art.

It will be apparent from the foregoing that, while particular forms of the invention have been illustrated an described, various modifications can be made without departing from the spirit and scope of the invention. Moreover, those skilled in the art will recognize that features shown in one embodiment may be utilized in other embodiments.

I claim:

1. A guidewire torque device for gripping and advancing a guidewire, comprising:
a main housing having a distal end, a proximal end and a guidewire lumen extending therethrough;
a first gear housing positioned within the main housing and having a first gear;
a second gear housing positioned within the main housing and having second gear;
the first gear being configured for slidable engagement and disengagement with the second gear;
a collet having a plurality of fingers for gripping a guidewire;
a collet housing having a distal end, a proximal end, and a lumen extending therethrough for receiving the collet;
a cap for engaging the collet and compressing the fingers of the collet when the cap is attached to the collet housing, the cap configured to receive an end of a guidewire and including a lumen through which the guidewire can pass;
a spring housing removably attached to the main housing and configured for retaining a spring therein; and
an actuator slidably positioned in the main housing is configured for moving the second gear housing into and out of engagement with the collet housing to simultaneously torque the guidewire and advance the guidewire distally relative to the guidewire torque device.

2. The guidewire torque device of claim 1, wherein the collet housing further comprises male threads proximate to the collet housing distal end and the cap has female threads proximate to the cap proximal end so that the cap can be screwed onto the collet housing.

3. The guidewire torque device of claim 2, wherein the collet comprises multiple substantially parallel, longitudinally-extending fingers.

4. The guidewire torque device of claim 3, wherein the actuator has a distal end and a proximal end, the distal end of the actuator being attached to the first gear housing.

5. The guidewire torque device of claim 4, wherein depressing the actuator distally moves the first gear into sliding engagement with the second gear.

6. The guidewire torque device of claim 5, wherein the main housing has grip bars to aid the physician in operating the torque device.

7. The guidewire torque device of claim 6, wherein the main housing has a plurality of grooves extending longitudinally on the main housing guidewire lumen.

8. The guidewire torque device of claim 7, wherein the main housing has male threads proximate to the distal end of the main housing.

9. The guidewire torque device of claim 8, wherein the first gear housing has a distal end and a proximal end.

10. The guidewire torque device of claim 9, wherein the first gear housing is sized for slidable engagement within the guidewire lumen of the main housing.

11. The guidewire torque device of claim 10, wherein the first gear housing has a first gear at the distal end of the first gear housing.

12. The guidewire torque device of claim 11, wherein the first gear has first gear teeth, the first gear teeth having first gear teeth ends that are angled relative to a longitudinal axis of the first gear teeth.

13. The guidewire torque device of claim 12, wherein the first gear teeth ends are angled in a range from 75° to 160°.

14. The guidewire torque device of claim 12, wherein the first gear teeth are spaced apart any of 20°, 30°, 45°, 60° or 75°.

15. The guidewire torque device of claim 14, wherein the second gear housing has a distal end, a proximal end, and a lumen extending therethrough.

16. The guidewire torque device of claim 15, wherein the second gear housing has second gear teeth, the second gear teeth having second gear teeth ends that are angled relative to a longitudinal axis of the second gear teeth.

17. The guidewire torque device of claim 16, wherein the second gear teeth ends are angled in a range from 75° to 160°.

18. The guidewire torque device of claim 17, wherein the second gear teeth are formed from elongated splines.

19. The guidewire torque device of claim 18, wherein the second gear housing has a non-round transverse cross-section.

20. The guidewire torque device of claim 19, wherein the non-round transverse cross-section can be any of a pentagon, a hexagon, or an octagon.

21. The guidewire torque device of claim 20, wherein longitudinal grooves are formed in corners of the pentagon, the hexagon, and the octagon.

22. The guidewire torque device of claim 21, wherein the collet housing has male threads proximate to the collet housing distal end.

23. The guidewire torque device of claim 22, wherein the collet housing has an outer tubular surface having longitudinally extending outer ridges.

24. The guidewire torque device of claim 23, wherein the collet housing is sized for slidable insertion into the second gear housing.

25. The guidewire torque device of claim 24, wherein the collet housing has a tapered portion at the collet housing distal end.

26. The guidewire torque device of claim 25, wherein the longitudinally extending outer ridges of the collet housing are sized for slidable engagement in the longitudinal grooves in the second gear housing.

27. The guidewire torque device of claim 26, wherein the plurality of fingers of the collet are spring biased toward an open position.

28. The guidewire torque device of claim 27, wherein the plurality of fingers on the collet have a gripping surface for gripping the guidewire.

29. The guidewire torque device of claim 28, wherein the cap has female threads so that the cap can be screwed onto the male threads of the collet housing.

30. The guidewire torque device of claim 29, the plurality of fingers being in a closed position when the cap is attached to the collet housing.

31. The guidewire torque device of claim 30, wherein the spring housing has a cavity for retaining the spring therein.

32. The guidewire torque device of claim 31, wherein the spring housing has female threads proximate to a spring housing distal end.

33. The guidewire torque device of claim 32, wherein the female threads of the spring housing engage the male threads of the main housing when the spring housing is removably attached to the main housing.

34. A guidewire torque device for gripping and advancing a guidewire, comprising:
a main housing having a distal end, a proximal end and a guidewire lumen extending therethrough;
a first gear housing positioned within the main housing and having a first gear;
a second gear housing slidably positioned within the main housing and having second gear;
the first gear being configured for slidable engagement and disengagement with the second gear;
a collet having a plurality of fingers for gripping a guidewire;
a collet housing having a distal end, a proximal end, and a lumen extending therethrough for receiving the collet;
a cap for engaging the collet and compressing the fingers of the collet when the cap is attached to the collet housing, the cap configured to receive an end of a guidewire and including a lumen through which the guidewire can pass;
a spring housing removably attached to the main housing and configured for retaining a spring therein; and
the second gear housing is advanced distally in the main housing thereby moving the second gear housing into engagement with the collet housing to simultaneously torque the guidewire and advance the guidewire distally relative to the guidewire torque device.

35. The guidewire torque device of claim 34, wherein the collet housing further comprises male threads proximate to the collet housing distal end and the cap has female threads proximate to the cap proximal end so that the cap can be screwed onto the collet housing.

36. The guidewire torque device of claim 35, wherein the collet comprises multiple substantially parallel, longitudinally-extending fingers.

37. The guidewire torque device of claim 36, wherein an actuator has a distal end and a proximal end, the distal end of the actuator being attached to the first gear housing.

38. The guidewire torque device of claim 37, wherein depressing the actuator distally moves the first gear housing distally and hence moves the first gear into sliding engagement with the second gear.

39. The guidewire torque device of claim 38, wherein the main housing has grip bars to aid the physician in operating the torque device.

40. The guidewire torque device of claim 39, wherein the main housing has a plurality of grooves extending longitudinally on the main housing guidewire lumen.

41. The guidewire torque device of claim 40, wherein the main housing has male threads proximate to the distal end of the main housing.

42. The guidewire torque device of claim 41, wherein the first gear housing has a distal end and a proximal end.

43. The guidewire torque device of claim 42, wherein the first gear housing is sized for slidable engagement within the guidewire lumen of the main housing.

44. The guidewire torque device of claim 43, wherein the first gear housing has a first gear at the distal end of the first gear housing.

45. The guidewire torque device of claim 44, wherein the first gear has first gear teeth, the first gear teeth having first gear teeth ends that are angled relative to a longitudinal axis of the first gear teeth.

46. The guidewire torque device of claim 45, wherein the first gear teeth ends are angled in a range from 75° to 160°.

47. The guidewire torque device of claim 45, wherein the first gear teeth are spaced apart any of 20°, 30°, 45°, 60° or 75°.

48. The guidewire torque device of claim 47, wherein the second gear housing has a distal end, a proximal end, and a lumen extending therethrough.

49. The guidewire torque device of claim 48, wherein the second gear housing has second gear teeth, the second gear teeth having second gear teeth ends that are angled relative to a longitudinal axis of the second gear teeth.

50. The guidewire torque device of claim 49, wherein the second gear teeth ends are angled in a range from 75° to 160°.

51. The guidewire torque device of claim 50, wherein the second gear teeth are formed from elongated splines.

52. The guidewire torque device of claim 51, wherein the second gear housing has a non-round transverse cross-section.

53. The guidewire torque device of claim 52, wherein the non-round transverse cross-section can be any of a pentagon, a hexagon, or an octagon.

54. The guidewire torque device of claim 53, wherein longitudinal grooves are formed in corners of the pentagon, the hexagon, and the octagon.

55. The guidewire torque device of claim 54, wherein the collet housing has male threads proximate to the collet housing distal end.

56. The guidewire torque device of claim 55, wherein the collet housing has an outer tubular surface having longitudinally extending outer ridges.

57. The guidewire torque device of claim 56, whereby the collet housing is sized for slidable insertion into the second gear housing.

58. The guidewire torque device of claim 57, wherein the collet housing has a tapered portion at the collet housing distal end.

59. The guidewire torque device of claim 58, wherein the longitudinally extending outer ridges of the collet housing are sized for slidable engagement in the longitudinal grooves in the second gear housing.

60. The guidewire torque device of claim 59, wherein the plurality of fingers of the collet are spring biased toward an open position.

61. The guidewire torque device of claim 60, wherein the plurality of fingers on the collet have a gripping surface for gripping the guidewire.

62. The guidewire torque device of claim 61, wherein the cap has female threads so that the cap can be screwed onto the male threads of the collet housing.

63. The guidewire torque device of claim 62, the plurality of fingers being in a closed position when the cap is attached to the collet housing.

64. The guidewire torque device of claim 63, wherein the spring housing has a cavity for retaining the spring therein.

65. The guidewire torque device of claim 64, wherein the spring housing has female threads proximate to a spring housing distal end.

66. The guidewire torque device of claim 65, wherein the female threads of the spring housing engage the male threads of the main housing when the spring housing is removably attached to the main housing.

67. A method of using a guidewire torque device for gripping and advancing a guidewire, comprising:
providing a main housing having a distal end, a proximal end and a lumen extending therethrough;
inserting a first gear housing within the main housing lumen, the first gear housing having a first gear;
inserting a second gear housing within the main housing lumen, the second gear housing having second gear;
the first gear being configured for slidable engagement and disengagement with the second gear;
providing a collet having a plurality of fingers for gripping a guidewire;
inserting the collet into a collet housing having a distal end, a proximal end, and a lumen extending therethrough for receiving the collet;
attaching a cap to the collet housing thereby compressing the fingers of the collet onto a guidewire;
inserting a spring into a spring housing and removably attaching the spring housing to the main housing; and
moving an actuator slidably positioned in the main housing for moving the second gear housing distally into engagement with the collet housing to simultaneously torque the guidewire and advance the guidewire distally relative to the guidewire torque device.

68. The method of claim 67, wherein the cap is screwed onto the collet housing.

69. The guidewire torque device of claim 68, wherein the plurality of fingers of the collet are biased toward an open position.

70. The guidewire torque device of claim 69, wherein depressing the actuator distally moves the first gear into sliding engagement with the second gear.

71. The guidewire torque device of claim 70, wherein slidably inserting the first gear housing into the lumen of the main housing.

72. The guidewire torque device of claim 71, wherein the first gear has first gear teeth, the first gear teeth having first gear teeth ends that are angled at 135° relative to a longitudinal axis of the first gear teeth, and wherein the second gear housing has second gear teeth, the second gear teeth having second gear teeth ends that are angled 135° relative to a longitudinal axis of the second gear teeth, further comprising distally advancing the first gear teeth into sliding engagement with the second gear teeth to thereby rotate the second gear housing and hence rotate the guidewire and simultaneously move the guidewire distally.
